(19) United States
(12) Patent Application Publication
(10) Pub. No.: US 2014/0341859 A1
(43) Pub. Date: Nov. 20, 2014

(54) METHOD FOR TREATMENT OF DAMAGED SITE OF BONE

(71) Applicants: TEIKYO UNIVERSITY, Tokyo (JP); GC CORPORATION, Tokyo (JP)

(72) Inventors: Katsuyuki YAMANAKA, Tokyo (JP); Tadashi KANEKO, Tokyo (JP); Yuuhiro SAKAI, Tokyo (JP); Yoshinobu WATANABE, Tokyo (JP); Takashi MATSUSHITA, Tokyo (JP); Satoshi ABE, Tokyo (JP)

(21) Appl. No.: 13/894,662

(22) Filed: May 15, 2013

(51) Int. Cl. A61K 35/32

(52) U.S. Cl. CPC A61K 35/32
USPC 424/93.7

(57) ABSTRACT

Provided is a treatment method which enables regeneration of bone tissue even when the size of a damaged site of bone is large. The treatment method comprises the steps of: culturing chondrocytes which have been seeded onto a porous body, or differentiating stem cells having chondrogenic differentiation potential which have been seeded onto a porous body into chondrocytes and culturing the chondrocytes; and implanting the porous body having the cultured chondrocytes.

### TECHNICAL FIELD OF THE INVENTION

The present invention relates to a method for repair and treatment of a damaged site of bone due to a bone-related disease, an accident, or the like.

### BACKGROUND ART

Many approaches employing so-called regenerative medicine have been made to treat a damaged site of bone such as a bone fracture or a bone defect due to a bone-related disease, an accident, or the like. The regenerative medicine is a medical technology for reproducing biotissue that can no longer recover by the healing capability of the living body, by using a cell, a cell carrier, a cell growth factor and so on, so that it has the same form and function as those of the original tissue.

In a treatment of bone employing such a regenerative medicine, a transplantation of autologous bone is most preferred. However, in a case that a damaged site of bone is large, since it is difficult to prepare and apply the autologous bone, the use of an artificial bone substitute material is required. Examples of the bone substitute material include hydroxyapatite and calcium phosphate complex; and implantation of these bone substitute material in combination with a base material having excellent biocompatibility and bioabsorbability has been performed.

In this respect, techniques as in Patent Documents 1 and 2 for example are disclosed from a viewpoint that the transplantation using autologous bone is most preferred as mentioned above.

Patent Document 1 (Japanese Patent Application Laid-Open No. 2006-116212) describes a sheet for guiding regeneration of mesenchymal tissue, wherein mesenchymal tissue precursor cells differentiated from mesenchymal cells and an extracellular matrix are adhered onto a porous sheet.

Patent Document 2 (Japanese Patent Application Laid-Open No. 2003-275294) discloses a bone regenerating sheet made by laminating a cultured cell sheet formed by culturing mesenchymal stem cells in a sheet shape and a biodegradable sheet made of biodegradable substances shaped into a sheet.

### SUMMARY OF THE INVENTION

However, the sheets described in Patent Documents 1 and 2 are used in such a way that the sheet adhered with cultured osteoblasts is implanted into the living body, thereby forming cortical bone from the osteoblasts through membranous ossification in the living body. According to this method, the osteoblasts cannot be cultured in a laminate manner. Therefore, such a sheet using an osteoblast layer cannot comprise a cell layer having a thickness of over 100 μm. As such, it is difficult to treat a large damaged site with the sheets.

Accordingly, an object of the present invention is to provide a method for treatment of a damaged site of bone which enables regeneration of bone tissue even when the size of the damaged site of bone is large.

The present invention will be described below.

A first aspect of the present invention is a method for treatment of a damaged site of bone, comprising the steps of: culturing chondrocytes which have been seeded onto a porous body, or differentiating stem cells having chondrogenic differentiation potential which have been seeded onto a porous body into chondrocytes and culturing the chondrocytes; and implanting the porous body having the cultured chondrocytes.

A second aspect of the present invention is the method for treatment of a damaged site of bone according to the first aspect, wherein the stem cells which differentiate into chondrocytes are mesenchymal stem cells.

A third aspect of the present invention is the method for treatment of a damaged site of bone according to the second aspect, wherein the mesenchymal stem cells have been harvested from at least one of human bone marrow, human synovium, human fat, and human periosteum.

A fourth aspect of the present invention is the method for treatment of a damaged site of bone according to the first aspect, wherein the chondrocytes which have been seeded onto a porous body are based on chondrocytes which have been harvested from another person or an animal; the stem cells having chondrogenic differentiation potential which have been seeded onto a porous body are based on stem cells having chondrogenic differentiation potential which have been harvested from another person or an animal; and the chondrocytes harvested and the stem cells harvested have been subjected to an antigen removal treatment such as decellularization through rapid freezing by liquid nitrogen.

A fifth aspect of the present invention is the method for treatment of a damaged site of bone according to the first aspect, wherein the porous body is formed of a polymer of lactic acid, glycolic acid or caprolactone, or a copolymer thereof.

A sixth aspect of the present invention is the method for treatment of a damaged site of bone according to the first aspect, comprising the step of pulverizing the porous body having the cultured chondrocytes into a powder form, wherein the implantation step is a step of implanting the porous body that has been made into a powder form in the pulverizing step.

According to the present invention, bone is regenerated through the ossification of chondrocytes and the surrounding bone formation promoting effects, and therefore it is possible to provide a treatment that regenerates bone tissue even when the size of the damaged site of bone is large.

### BRIEF DESCRIPTION OF THE DRAWINGS

FIG. 1 is a view illustrating a placement step in a method S10 for treatment of a damaged site of bone.

FIG. 2 is a view illustrating a cell suspension introduction step in the method S10 for treatment of a damaged site of bone.

FIG. 3 is a view illustrating a situation, in the method S10 for treatment of a damaged site of bone, where an entire porous body is filled up with the cell suspension.

FIG. 4 is a view illustrating a reversing step in the method S10 for treatment of a damaged site of bone.

FIG. 5 is a view illustrating a temporary placement step in a method S20 for treatment of a damaged site of bone.

FIG. 6 is a view illustrating a situation, in the method S20 for treatment of a damaged site of bone, where an entire porous body is filled up with the cell suspension.

FIG. 7 is a view illustrating a sandwiching step in the method S20 for treatment of a damaged site of bone.

FIG. 8 is a view illustrating a holding step in the method S20 for treatment of a damaged site of bone.

### DETAILED DESCRIPTION OF THE INVENTION

The functions and benefits of the present invention will be apparent from the following modes for carrying out the invention. However, the present invention is not limited to these embodiments.

First, the method S10 for treatment of a damaged site of bone according to one embodiment (hereinafter sometimes referred to as “treatment method S10”) will be described.

Examples of medical applications which the present invention may be adopted include:

1) treatment of large bone defect that cannot be dealt with by general donor bone transplantation;
2) treatment of non-union or delayed union after a bone fracture;
3) treatment of bone defect associated with osteomyelitis or infectious non-union;
4) enhancement of bone union after a bone lengthening;
5) reconstruction of a fractured bone associated with a bone defect;
6) treatment of bone defect after resection of bone tumor;
7) anterior spinal fusion;
8) posterior spinal fusion;
9) posterolateral spinal fusion;
10) bone filling in an artificial joint revision surgery;
11) bone implantation into a bone defect in a donor site.

In this way, the present invention can be applied in regions where autologous bone transplantation surgery or osteogenic protein has been conventionally employed for promoting bone formation and can be applied in cases where autologous bone transplantation, or bone filling or reinforcement by an artificial bone or metal is needed for bone defects and the like. At this time, the present invention may be applied alone or in combination with the conventional methods.

Further, in the dental field as well, the present invention is applicable to such cases as bone augmentation to compensate for bone loss, sinus lift, or socket lift at the time of embedding an implant, and bone filling at the time of removing jawbone cyst. However, the present invention is not limited to these cases.

The treatment method S10 comprises: a cell harvesting step S11; an expansion culture step S12; a cell suspension preparation step S13; a tissue regenerating material production step S14; a cartilage tissue culture step S15; and an implantation step S16. Each of them are described below.

The cell harvesting step S11 is a step of obtaining cells that serve as a source to finally form bone cells. The cells to be harvested are chondrocytes or stem cells having chondrogenic differentiation potential (hereinafter, the stem cell sometimes referred to as a “chondrocyte differentiation stem cell”). As to the chondrocytes and the chondrocyte differentiation stem cells that are harvested, in the case of the chondrocytes, they may be used directly, and in the case of the chondrocyte differentiation stem cells, the following may be used: stem cells such as a bone marrow-derived mesenchymal stem cell, a fat-derived mesenchymal stem cell, a mesenchymal cell, and a synovial cell, which can differentiate into chondrocytes or can promote restoration thereof. Examples thereof include cells harvested from a bone marrow of a pelvis (an iliac bone) or of long bones of arms and legs (a thighbone, a tibia) and/or a bone marrow of periosteum, synovium, fat, alveolar bone or the like, a periosteum of a palate or an alveolar bone, and so on.

As a way to harvest these, methods ordinarily carried out in the medical setting may be employed without particular limitations. Among these, such sources as the bone marrow of the iliac bone or the like, and the periosteum of the palate, the alveolar bone or the like are preferably employed as they enable an easy operation with a minimum exfoliation and incision of the skin and the muscle at the time of harvesting.

Further, the chondrocytes and the chondrocyte differentiation stem cells to be harvested may be harvested not only from a patient to which the treatment method S10 is applied, but also from another person dead or alive, or an animal such as a cow, pig, horse, and chicken. However, when taking the cells from another person or an animal, in consideration of the possibility that the immunological rejection may occur, it is necessary to carry out, in the final procedure, an antigen removal treatment such as decellularization through rapid freezing by liquid nitrogen. According to this, the burden on the patient to be implanted can be reduced.

The expansion culture step S12 is a step of culturing to amplify the chondrocytes or the chondrocyte differentiation stem cells that have been harvested, for one to two weeks using a culture dish for tissue culture by a general method. The culture medium to be used for expansion culture may be a known one; preferably, αMEM containing autologous serum or fetal bovine serum may be suitably employed. At this time, in the case of the mesenchymal stem cells, when a specific growth factor (for example, bFGF) is activated, the mesenchymal stem cells are proliferated while keeping a high multiple differentiation potency, enabling facilitation of chondrogenic differentiation.

The cell suspension preparation step S13 is a step of preparing a cell suspension which contains the cells that have been cultured and amplified in the expansion culture step S12. In specific, the cell suspension is prepared by suspending the stem cells into a culture medium for chondrogenic differentiation. The culture medium to be used herein, that is, the culture medium for chondrogenic differentiation may be a known one. The cell suspension to be prepared preferably has a cell concentration of 5×10⁶ to 1×10⁸ cells/ml.

The tissue regenerating material production step S14 is a step of producing a tissue regenerating material by introducing the cell suspension prepared in the cell suspension preparation step S13 into a porous body to seed and adhere the cells thereto. Herein, the tissue regenerating material refers to a porous body seeded with the cells.

The porous body to be used herein is made of a bioabsorbable polymer material; has communicating pores with a pore diameter of 180 to 3500 μm and an average pore diameter of 350 to 2000 μm; and has a porosity of 60 to 95%. In addition, the compressive strength of the porous body is arranged to be 0.05 to 1 MPa. Herein, the pore diameter of the porous body does not take into account micropores of less than 10 μm that only liquid can pass therethrough; and means that 80% or more of the pores of 10 μm or more in the entire porous body have a pore diameter of 180 to 3500 μm. The “porosity” is a value calculated from the weight of the porous body with respect to the weight of a lump of raw material of the bioabsorbable polymer material used, the porous body having the same volume as that of the lump of raw material of the bioabsorbable polymer material used.

If the porosity is less than 60%, efficiency of culturing the chondrocytes or the chondrocyte differentiation stem cells decreases. If it exceeds 95%, the strength of the porous body itself degrades. Therefore, the porosity is preferably 80 to 90%. In addition, if the pore diameter is less than 180 μm, it is difficult to introduce the chondrocytes or the chondrocyte differentiation stem cells into the porous body, making it impossible to seed the chondrocytes or the chondrocyte differentiation stem cells sufficiently into the porous body. On the other hand, if the pore diameter is more than 3500 μm, the strength of the porous body itself degrades.

Further, the average pore diameter is preferably 540 to 1200 μm.

As for the above compressive strength, if the compressive strength is less than 0.05 MPa, the porous body shrinks due to the extension stress of the chondrocytes or the chondrocyte differentiation stem cells. On the other hand, it is technically difficult to make a porous body having a compressive strength of over 1 MPa. The “compressive strength” refers to compressive fracture strength generated at a time of compressing a cylindrical test piece in a size of 10 mm diameter×2 mm height at a cross head speed of 1 mm/min.

The shape of the porous body as a whole is not particularly limited; however, it may be a shape that corresponds to a shape of a damaged site to be implanted. However, common porous bodies of various basic shape such as a cube, cuboid, hemisphere, and circular plate may be employed. The thickness of the porous body (the size in the vertical direction at the time of culturing) is preferably 2 to 100 mm, more preferably 2.1 to 70 mm. If the thickness exceeds 100 mm, it is difficult to introduce the cells, or culture the cells for a long period.

The material to constitute the porous body may be any without particular limitations as long as it is a bioabsorbable polymer material and can maintain its configuration in the body for a certain period. For example, at least one selected from the followings that have been conventionally used may be employed: polyglycolic acid; polylactic acid; a copolymer of lactic acid and glycolic acid; poly-ε-caprolactone; a copolymer of lactic acid and ε-caprolactone; polyamino acid; polyortho ester; and a copolymer thereof. Among these, polyglycolic acid, polylactic acid, and a copolymer of lactic acid and glycolic acid are most preferred as being approved by US Food and Drug Administration (FDA) as a polymer harmless to the human body and in view of their actual performance. The weight average molecular weight of the bioabsorbable polymer material is preferably 5000 to 2000000, and more preferably 10000 to 500000.

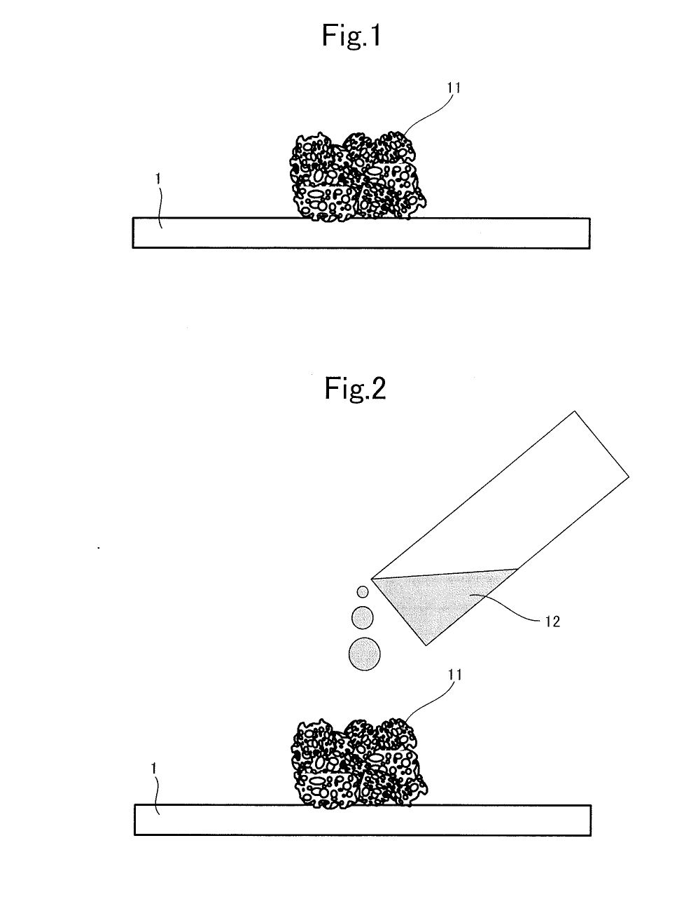

By using such a porous body, the cell suspension can properly permeate into the porous body 11 (Refer to FIG. 1), and the cells can be introduced into the porous body to be seeded therein stably without being wasted, thereby enabling production of the tissue regenerating material.

The production method of the porous body is not particularly limited. However, the production method may be for example as follows: mixing, in a substantially uniform manner, a particulate substance having a particle diameter of 100 to 2000 μm into a solution having a bioabsorbable polymer material dissolved in an organic solvent and freezing the mixture, the particulate substance not dissolving in this organic solvent but dissolving in a liquid that does not dissolve the bioabsorbable polymer material; thereafter drying the mixture to remove the organic solvent; thereby producing a porous bioabsorbable polymer which has a porous structure with a pore diameter of 5 to 50 μm and contains the particulate substance; pulverizing this porous bioabsorbable polymer with a mill or the like; then removing the particulate substance by dissolving it in the liquid that does not dissolve the bioabsorbable polymer; thereafter sifting it to make a bioabsorbable granular porous material having an average particle size of 100 to 3000 μm; and then putting the bioabsorbable granular porous material into a container having a predetermined shape to pressurize and heat it.

The tissue regenerating material production step S14 comprises: a placement step S141; a cell suspension introduction step S142; and a reversing step S143. FIGS. 1 to 4 illustrate the tissue regenerating material production step S14.

The placement step S141 is a step of placing the porous body 11 on a holding plate 1, as shown in FIG. 1. Herein, the holding plate 1 is preferably a plastic plate or a glass plate having a contact angle to water of 15° to 90°. This holding plate 1 can be obtained by processing into a plate shape a glass material used for a petri dish, flask, multiwell, etc., which have been conventionally used as a container for static culture of cells, or a plastic material such as polystyrene, polyethylene, and polyethylene terephthalate, etc. These materials sometimes have a high hydrophobic property. Therefore, it is preferable to perform, as necessary, a chemical treatment such as a plasma (corona discharge) treatment on the surface to be contacted with the porous body 11 to introduce a polar group and increase its hydrophilicity and to make the surface have a contact angle to water of 15° to 90°. Further, a ceramic coating may be formed on the surface to be contacted. It is preferable for the surface to be contacted to be smooth; however, it may have grooves or streaky protrusions with a height of several hundred micrometers.

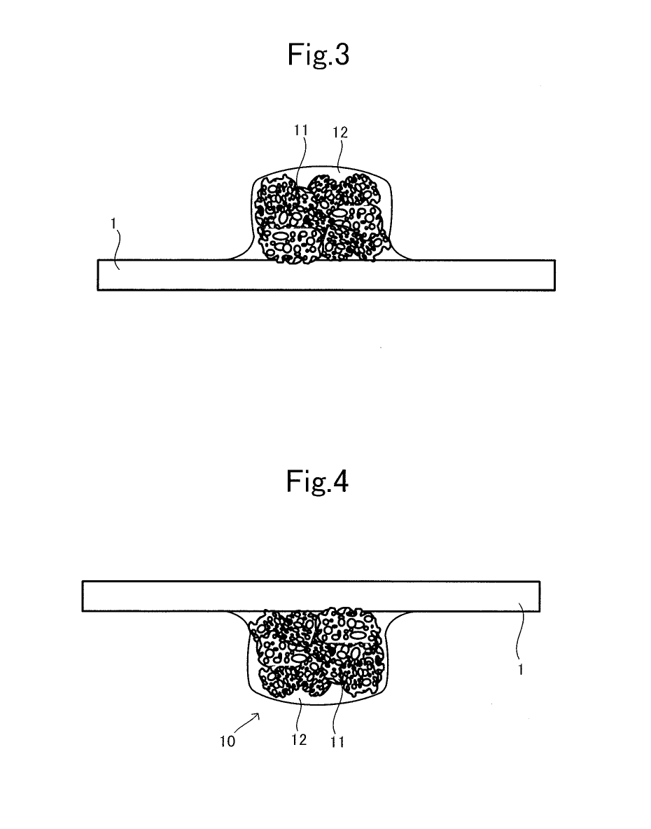

Through the placement step S141, the porous body 11 is arranged on the surface of the holding plate 1. Then in the cell suspension introduction step S142, the cell suspension 12 prepared in the cell suspension preparation step S13 is introduced into the porous body 11 for example by dripping or injection, as shown in FIG. 2. By this, the cell suspension 12 permeates into the porous body 11 and fills up the entire porous body 11, as shown in FIG. 3. At this time, if the amount of the cell suspension 12 is too large in relation to the volume of the porous body 11, it is likely to be difficult to carry out the below described reversing step S143 or the culture efficiency tends to degrade. Therefore, it is preferable to introduce the cell suspension in an amount that can immerse the entire porous body 11 and that does not leak excessively from the porous body 11.

Next, through the reversing step S143, the holding plate 1 and the porous body 11 containing the cell suspension 12 are reversed from the state shown in FIG. 3, so that the holding plate 1 is on the porous body 11, as shown in FIG. 4. That is, when seen in the gravity direction, the holding plate 1 is on the upper side of the porous body 11, and they are kept still in the air in a state that the weight of the holding plate 1 is not applied to the porous body 11. With the holding plate 1 and the porous body 11 kept still in this state, the cells that have been introduced are adhered to the inner walls of the pores in the porous body 11, thereby completing seeding thereof. The obtained is the tissue regenerating material 10. The time to keep the holding plate 1 and the porous body 11 still in the air in order to adhere the cells to the porous body varies depending on the material of the porous body and the kinds of cells to be seeded; however, it is generally 20 to 300 minutes.

Further, when making the tissue regenerating material with the porous body positioned under the holding plate as in the present embodiment, the shape of the porous body is not particularly limited as long as the holding plate 1 can be arranged on the upper side through the reversing step S143 when seen in the gravity direction, the porous body can be kept still in the air in the state that the weight of the holding plate 1 is not applied to the porous body 11, and the cells can be seeded therein. However, at this time the area of the base (the area of the face which contacts the holding plate 1) of the porous body 11 is preferably large enough in relation to the thickness thereof. In specific, the area of the base is preferably 0.5 to 200 cm² in terms of the range of the volume of 1 to 50000 cm³ of the porous body. If the area of the base is less than 0.5 cm² or if it exceeds 200 cm², it is difficult to carry out the seeding with the porous body hung on the holding plate.

Herein, if the porous body 11 has a relatively large hole, the cell suspension 12 can properly permeate into the porous body 11. And finally the cell suspension 12 can be kept in a way not leaking from the porous body 11, by arranging the holding plate 1 on the porous body 11 as above when seen in the gravity direction and keeping them still in the air in the state that the weight of the holding plate 1 is not applied to the porous body 11. Accordingly, it is possible to introduce the cell suspension 12 stably without wasting it and seed the cells into the porous body.

The cartilage tissue culture step S15 is a step of making a bone regenerating cartilage tissue material from the tissue regenerating material 10 produced in the tissue regenerating material production step S14. In the cartilage tissue culture step S15, when the cells seeded in the tissue regenerating material 10 are the chondrocytes, these are cultured to be amplified. If the cells seeded in the tissue regenerating material 10 are the chondrocyte differentiation stem cells, they are differentiated into the chondrocytes, which are cultured to be amplified.

Therefore, the bone regenerating cartilage tissue material is formed from the tissue regenerating material 10, comprising the porous body after the culturing process and the chondrocytes cultured and contained in this porous body. That is, the bone regenerating cartilage tissue material is made by culturing the chondrocytes or the chondrocytes that have differentiated from the chondrocyte differentiation stem cells that are contained in the above described tissue regenerating material.

The expansion/differentiation (culturing) of these chondrocytes may be carried out by a known method. For example, a proliferation culture medium may be used to amplify them. Also, after they are proliferated, they may be differentiated and cultured by using a culture medium for chondrogenic differentiation.

The implantation step S16 is a step of implanting the bone regenerating cartilage tissue material made in the cartilage tissue culture step S15 into a site of the bone damaged by a bone-related disease or an accident. This enables bone regeneration in the body through enchondral ossification. On the other hand, since the porous body is formed of a bioabsorbable polymer material, it disappears with time.

Next, a method S20 for treatment of a damaged site of bone according to another embodiment (hereinafter sometimes referred to as a “treatment method S20”) will be described. In the present embodiment, the same elements as those described in the above treatment method 10 are given the same numerals, and descriptions thereof will be omitted.

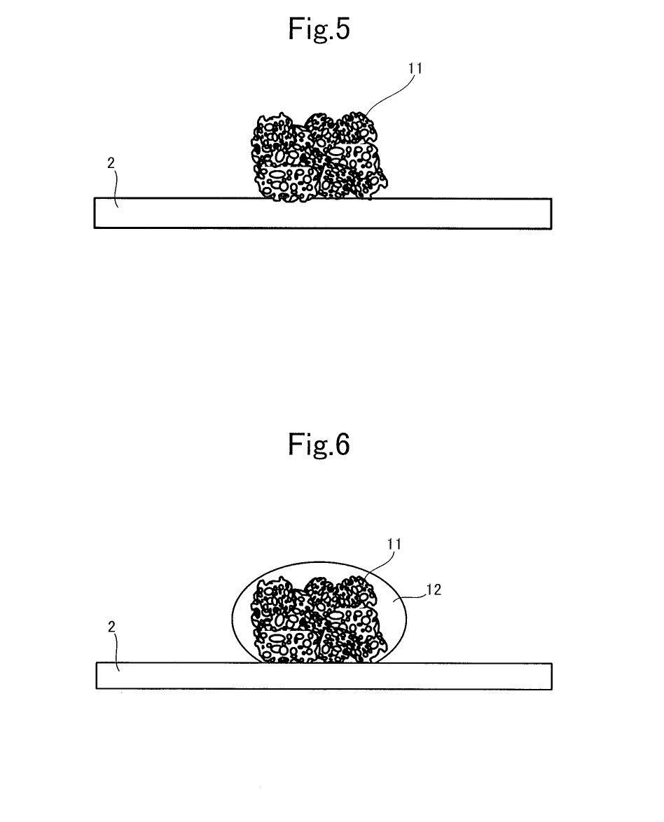

The treatment method S20 comprises a tissue regenerating material production step S24 instead of the tissue regenerating material production step S14 of the treatment method S10. The tissue regenerating material production step S24 comprises: a temporary placement step S241; a cell suspension introduction step S242; a sandwiching step S243; and a holding step S244. FIGS. 5 to 8 illustrate the tissue regenerating material production step S24.

The temporary placement step S241 is a step of placing the porous body 11 onto a temporary placement plate 2, which is a plate member having high water repellency, as shown in FIG. 5. The temporary placement plate 2 is preferably a plastic plate or a glass plate having a contact angle to water of larger than 90°.

In the cell suspension introduction step S242, the cell suspension 12 is introduced into the porous body 11, as in the above-described cell suspension introduction step S142. Through this, the cell suspension 12 permeates into the porous body 11 to fill up the entire porous body 11, as shown in FIG. 6.

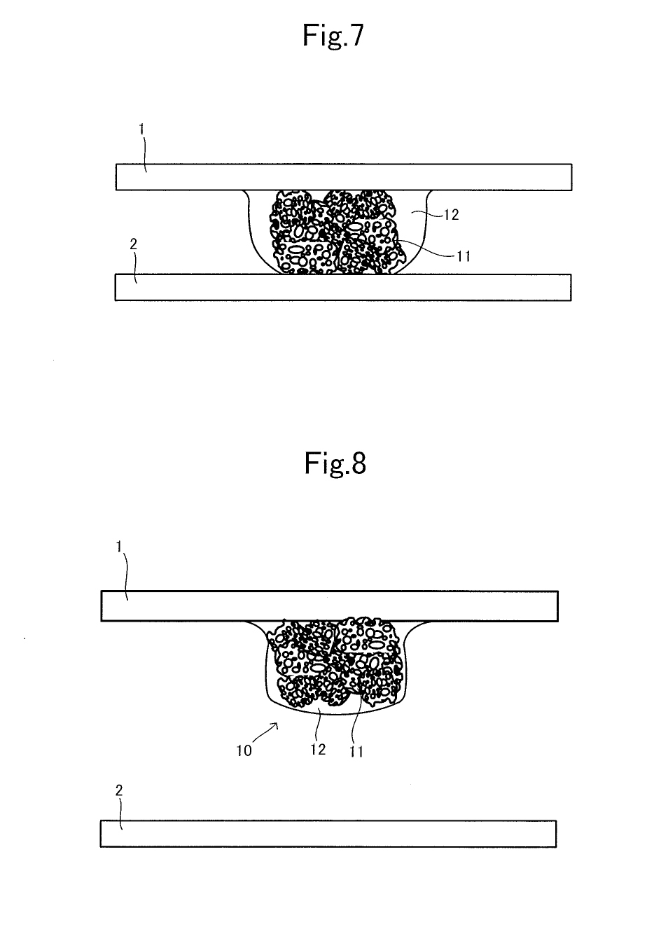

The sandwiching step S243 is a step of contacting the porous body 11 filled up with the cell suspension to a face of the holding plate 1 in a manner sandwiching the porous body 11 between the holding plate 1 and the temporary placement plate 2, as shown in FIG. 7.

The holding step S244 is a step of pulling up the holding plate 1 contacted with the porous body 11, letting the porous body 11 be held on the holding plate 1 side, and releasing it from the temporary placement plate 2, as shown in FIG. 8. Through this, as in the above one embodiment, the holding plate 1 is arranged to be on the upper side of the porous body 11 when seen in the gravity direction, and they are kept still in the air in a state that the weight of the holding plate 1 is not applied to the porous body 11. With the holding plate 1 and the porous body 11 kept still in this state, the cells that have been introduced are adhered to an inner wall of the porous body 11, thereby completing seeding thereof. The obtained is the tissue regenerating material 10.

Herein, the temporary placement plate 2 is constituted by a water-repellent plate; and the holding plate 1 is constituted by a hydrophilic plate. Therefore, the above described holding and releasing of the porous body filled with the cell suspension can be properly done.

According to the treatment methods S10 and S20 as described above, it is possible to make a cartilage having a large volume, and to make a bone having a large volume from this through enchondral ossification. Accordingly, even when the size of the damaged site of bone is large, it is possible to regenerate bone within a short time by implanting this cartilage. That is, a large cartilage in bulk form equivalent to the size of the porous body is made and implanted into the damaged site, thereby regenerating the bone having a large volume through enchondral ossification. Further, the cartilage may be implanted after pulverizing the cartilage in bulk form into a powder form to make it easily applicable to the implantation site.

Examples will be described below.

### Example 1

In Example 1, a bone regenerating cartilage tissue material D1 was made through the following process, and implanted through the implantation step.

(Preparation of Cell A1 Through the Cell Harvesting Step and the Expansion Culture Step)

The cells taken from a human iliac bone marrow were suspended in αMEM culture medium with a 20% FBS at a concentration of 1×10⁴ cells/ml nucleated cells. Thereafter, 10 ml thereof was seeded in a culture dish having a diameter of 10 cm. The cells were cultured to be proliferated under the presence of 5% carbon dioxide gas at 37° C. Three days after seeding, floating cells (hematopoietic cells) were removed and the medium was replaced by fresh medium. After that, the culture medium was changed every three days. From the fifth day, bFGF was added to the culture medium in an amount of 3 ng/ml. Around the 10th day, the cells were proliferated to be nearly confluent. The culture dish was incubated for 5 minutes with trypsin (0.05%)-EDTA (0.2 mM); and the cells were isolated. The number of the cells was measured by a Coulter counter (ZI single, manufactured by Coulter Corporation), and the cells were seeded at a density of 5000 cells/cm². The third passage cells were used which were obtained from the second subculture dish made nearly confluent by repeating this operation.

Thereby, the cells A1, which were the bone marrow derived mesenchymal stem cells taken from the human iliac bone marrow, were obtained.

(Preparation of Porous Body B1)

A DL-lactic acid/glycolic acid copolymer having a molecular weight of 250000 was dissolved in dioxane, and mixed with sodium chloride having a particle size of around 500 μm. Then the mixture was freeze-dried, and was pulverized and desalinated in order to obtain a powder formed material. The powder formed material thus obtained was shaped by compression heating and γ-sterilized. Thereby, a porous body in a disc block shape was made which was composed of a bioabsorbable synthetic polymer and had an average pore diameter of 540 μm, a porosity of 90%, a compressive strength of 0.2 MPa, a diameter of 9 mm and a thickness of 3 mm. In this way, a porous body B1 of a block of a polylactic acid/glycolic acid copolymer (PLGA) was obtained.

(Production of Tissue Regenerating Material C1 Through the Cell Suspension Preparation Step and the Tissue Regenerating Material Production Step)

The disc-shaped bottom face of the above porous body B1 was contacted and placed onto an upper surface of a 60 mm culture dish of plasma treated polystyrene (with a contact angle to water of 70°). The above cells A1, which were suspended in a chondrogenic differentiation medium (αMEM, glucose 4.5 mg/ml, 10⁻⁷ M dexamethasone, 50 μg/ml ascorbic acid-2-phosphoric acid, 10 ng/ml TGF-β, 6.25 μg/ml insulin, 6.25 μg/ml transferrin, 6.25 ng/ml selenic acid, 5.33 μg/ml linoleic acid, 1.25 mg/ml bovine serum albumin) at a concentration of 2×10⁷ cells/ml, were seeded into the porous body B1 in an amount of 0.19 ml by dripping. Then with the culture dish reversed to be upside down, the cells A1 were seeded on the porous body B1 by adhering the cells to the material for 100 minutes under the conditions of 37° C., 100% humidity, and 5% CO₂, to thereby obtain the tissue regenerating material C1.

(Production of Bone Regenerating Cartilage Tissue Material D1 Through the Cartilage Tissue Culture Step)

Thereafter, the tissue regenerating material C1 was put into a 50 ml centrifuge tube filled with a chondrogenic differentiation medium, and was cultured at 37° C. for four weeks while changing the culture medium every three days. Thereby, the bone regenerating cartilage tissue material D1 was produced.

(Implantation by the Implantation Step and the Evaluation Thereof)

The bone regenerating cartilage tissue material D1 was implanted into subcutaneous area of the back of a SCID mouse; and the implant was collected after the eighth week to be evaluated with μCT and histopathologically. According to the results, it was confirmed that the implant was a mature bone tissue with a large amount of calcium deposited, and that the treatment method according to Example 1 enabled regeneration of ectopic bone.

### Example 2

In Example 2, a bone regenerating cartilage tissue material D2 was made through the following process, and implanted through the implantation step.

(Preparation of Stem Cell A2 Through the Cell Harvesting Step and the Expansion Culture Step)

A rat's femur and tibia were taken, and the bone marrow was flushed out to collect cells using a culture medium. The cells thus collected were suspended in αMEM culture medium with a 10% FBS at a concentration of 1×10⁴ cells/ml nucleated cells. Thereafter, 10 ml thereof was seeded in a culture dish having a diameter of 10 cm. The cells were cultured to be proliferated at 37° C. under the presence of 5% carbon dioxide gas. Changing the culture medium on the third day, non-adherent cells (hematopoietic cells) were removed. After that, the culture medium was changed every three days. From the fifth day, bFGF was added to the culture medium in an amount of 3 ng/ml. Around the 10th day, the cells were proliferated to be nearly confluent. The culture dish was incubated for 5 minutes with trypsin (0.05%)-EDTA (0.2 mM); and the cells were isolated. The number of the cells was measured by a Coulter counter (ZI single, manufactured by Coulter Corporation), and the cells were seeded at a density of 5000 cells/cm². The third passage cells were used which were obtained from the second subculture dish made nearly confluent by repeating this operation.

Thereby, the cells A2, which were the mesenchymal stem cells taken from the rat's femur and tibia bone marrow, were obtained.

(Preparation of Porous Body. B2)

A DL-lactic acid/glycolic acid copolymer having a molecular weight of 250000 was dissolved in dioxane, and thereafter mixed with sodium chloride having a particle size of around 500 μm. Then the mixture was freeze-dried, and was pulverized and desalinated to thereby obtain a powder formed material. The powder formed material thus obtained was shaped by compression heating and γ-sterilized. Thereby, a porous body in a block shape was made which was composed of a bioabsorbable synthetic polymer material and had an average pore size of 600 μm, a porosity of 80%, a compressive strength of 0.6 MPa, and a size of 3 mm×5 mm×20 mm. In this way, a porous body B2 of a block of a polylactic acid/glycolic acid copolymer (PLGA) was obtained.

(Production of Tissue Regenerating Material C2 Through the Cell Suspension Preparation Step and the Tissue Regenerating Material Production Step)

The bottom face of the porous body B2 was contacted and placed onto an upper surface of a 60 mm culture dish of plasma treated polystyrene (with a contact angle to water of 70°). The above cells A2, which were suspended in a culture medium for chondrogenic differentiation (nMEM, glucose 4.5 mg/ml, 10⁻⁷ M dexamethasone, 50 μg/ml ascorbic acid-2-phosphoric acid, 10 ng/ml TGF-β, 6.25 μg/ml insulin, 6.25 μg/ml transferrin, 6.25 ng/ml selenic acid, 5.33 μg/ml linoleic acid, 1.25 mg/ml bovine serum albumin) at a concentration of 2×10⁷ cells/ml, were introduced into the porous body B2 in an amount of 0.19 ml by dripping. With the culture dish reversed to be upside down, the cells A2 were seeded on the porous body B2 by adhering the cells to the material for 100 minutes under the conditions of 37° C., 100% humidity, and 5% CO₂, to thereby obtain the tissue regenerating material C2.

(Production of Bone Regenerating Cartilage Tissue Material D2 Through the Cartilage Tissue Culture Step)

Thereafter, the tissue regenerating material C2 was put into a 50 ml centrifuge tube filled with a chondrogenic differentiation cell fluid, and cultured at 37° C. for four weeks while changing the culture medium every three days. Thereby, the bone regenerating cartilage tissue material D2 was produced.

(Implantation by the Implantation Step and the Evaluation Thereof)

A rat femur was fixated by an external fixator and a bone defect with a gap of 5 mm was created. The model thus obtained was implanted with the bone regenerating cartilage tissue material D2, which was cut by a surgical knife into a size of 3 mm×3 mm×5 mm. A healing process of the defect site on the first, second, fourth, eighth and sixteenth weeks after the implantation was evaluated by an X-ray. Further, on the sixteenth week after the implantation, the femur was taken out to be evaluated with μCT and histopathologically. According to the results, it was confirmed that the implant was changed to a mature bone tissue with a large amount of calcium deposited, and that the treatment method according to Example 2 enabled a regenerative treatment of bone in the gap site of the femur.

### Example 3

In Example 3, a bone regenerating cartilage tissue material D3 was made through the following process, and implanted through the implantation step.

(Preparation of Stem Cell A3 Through the Cell Harvesting Step and the Expansion Culture Step)

The cells taken from a dog's iliac bone marrow were suspended in αMEM culture medium with a 20% FBS at a concentration of 1×10⁴ cells/ml nucleated cells. Thereafter, 10 ml thereof was seeded in a culture dish having a diameter of 10 cm. The cells were cultured to be proliferated under the presence of 5% carbon dioxide gas at 37° C. Changing the culture medium on the third day, non-adherent cells (hematopoietic cells) were removed. After that, the culture medium was changed every three days. From the fifth day, bFGF was added to the culture medium in an amount of 3 ng/ml. Around the 10th day, the cells were proliferated to be nearly confluent. The culture dish was incubated for 5 minutes with trypsin (0.05%)-EDTA (0.2 mM); and the cells were isolated. The number of the cells was measured by a Coulter counter (ZI single, manufactured by Coulter Corporation), and the cells were seeded at a density of 5000 cells/cm². The third passage cells were used which were obtained from the second subculture dish made nearly confluent by repeating this operation.

Thereby, the cells A3, which were the mesenchymal stem cells taken from the dog's iliac bone marrow, were obtained.

(Preparation of Porous Body B3)

A DL-lactic acid/glycolic acid copolymer having a molecular weight of 250000 was dissolved in dioxane, and thereafter mixed with sodium chloride having a particle size of around 500 μm. Then the mixture was freeze-dried, and was pulverized and desalinated to thereby obtain a powder formed material. The powder formed material thus obtained was shaped by compression heating and γ-sterilized. Thereby, a block porous body in a doughnut shape was made which was composed of a bioabsorbable synthetic polymer material and had an average pore size of 600 μm, a porosity of 80%, a compressive strength of 0.6 MPa, and a size of an outer diameter 14 mm×inner diameter 8 mm×30 mm. In this way, a porous body B3 of a block of a polylactic acid/glycolic acid copolymer (PLGA) was obtained.

(Production of Tissue Regenerating Material C3 Through the Cell Suspension Preparation Step and the Tissue Regenerating Material Production Step)

The disc-shaped bottom face of the porous body B3 was contacted and placed onto an upper surface of a 60 mm culture dish of plasma treated polystyrene (having a contact angle to water of 70°). The above cells A3, which were suspended in a culture medium for chondrogenic differentiation (αMEM, glucose 4.5 mg/ml, 10⁻⁷ M dexamethasone, 50 μg/ml ascorbic acid-2-phosphoric acid, 10 ng/ml TGF-β, 6.25 μg/ml insulin, 6.25 μg/ml transferrin, 6.25 ng/ml selenic acid, 5.33 μg/ml linoleic acid, 1.25 mg/ml bovine serum albumin) at a concentration of 2×10⁷ cells/ml, were introduced into the porous body B3 in an amount of 0.19 ml by dripping. With the culture dish reversed to be upside down, the cells A3 were seeded on the porous body B3 by adhering the cells A3 to the material for 100 minutes under the conditions of 37° C., 100% humidity, and 5% CO₂, to thereby obtain the tissue regenerating material C3.

(Production of Bone Regenerating Cartilage Tissue Material D3 Through the Cartilage Tissue Culture Step)

Thereafter, the tissue regenerating material C3 was put into a 50 ml centrifuge tube filled with a chondrogenic differentiation cell fluid, and cultured at 37° C. for four weeks while changing the culture medium every three days. Thereby, the bone regenerating cartilage tissue material D3 was produced.

(Implantation by the Implantation Step and the Evaluation Thereof)

A model which was obtained by creating a bone defect with a gap of 30 mm in a dog's femur and fixated with a plate, was implanted with the bone regenerating cartilage tissue material D3. A healing process of the defect site on the first, second, fourth, eighth and sixteenth weeks after the implantation was evaluated by an X-ray. Further, on the sixteenth week after the implantation, the femur was taken out to be evaluated with μCT and histopathologically. According to the results, it was confirmed that the implant was a mature bone tissue with a large amount of calcium deposited, and that the treatment method according to Example 3 enabled a regenerative and restorative treatment of bone in the large bone defect site of the femur.

### Example 4

In Example 4, a bone regenerating cartilage tissue material D2 was made and implanted through the implantation step. That is, in Example 4, the same bone regenerating cartilage tissue material D2 as in Example 2 was used.

(Implantation by the Implantation Step and the Evaluation Thereof)

The bone regenerating cartilage tissue material D2 was cut by a surgical knife into a size of 3 mm×3 mm×2 mm, and implanted under the buccal periosteum of the upper jaw in the molar region of a rat. On the eighth week after the implantation, the upper jaw bone was taken out to be evaluated with μCT and histopathologically. According to the results, it was confirmed that a mature bone tissue having a thickness of 2 mm which was continuous with the upper jaw bone grew in the upper area of the upper jaw bone. Thus it was confirmed that the treatment method according to Example 4 would enable regeneration and growth of alveolar bone and jaw bone of a patient whose jaw bone has been absorbed and who cannot be treated with a dental implant.

### Example 5

In Example 5, a bone regenerating cartilage tissue material D2 was made and implanted through the implantation step. That is, in Example 5, the same bone regenerating cartilage tissue material D2 as in Example 2 was used.

(Implantation by the Implantation Step and the Evaluation Thereof)

The bone regenerating cartilage tissue material D2 was cut by a surgical knife into a size of 3 mm×3 mm×5 mm, and implanted under the periosteum of a rat skull. On the eighth week after the implantation, the skull was taken out to be evaluated with μCT and histopathologically. According to the results, it was confirmed that a mature bone tissue having a thickness of 3 mm or more, which was continuous with the skull, grew in the upper area of the skull. It was confirmed that the treatment method according to Example 5 enabled bone regeneration in the skull and maxillofacial area.

### Example 6

In Example 6, a bone regenerating cartilage tissue material D2 was made and implanted through the implantation step. That is, in Example 6, the same bone regenerating cartilage tissue material D2 as in Example 2 was used.

(Implantation by the Implantation Step and the Evaluation Thereof)

A model obtained by creating a cylindrical osteochondral defect in a size of Ø2×10 mm in a rat's femur in a direction from the knee joint to the subarticular bone, was implanted with the bone regenerating cartilage tissue material D2 which was cut into a size of 2 mm×2 mm×10 mm by a surgical knife. On the eighth week after the implantation, the femur was taken out to be evaluated with μCT and histopathologically. According to the results, it was confirmed that the osteochondral defect site from the knee joint toward the center of the femur was regenerated and that a mature bone tissue was regenerated in a continuous manner so that the transition between the bone tissue and the surrounding tissue could not be identified. It was confirmed that the treatment method according to Example 6 enabled bone regeneration in the articular cartilage and subchondral bone.

### DESCRIPTION OF THE REFERENCE NUMERALS

1 holding plate
2 temporary placement plate
10 tissue regenerating material
11 porous body
12 cell suspension

1. A method for treatment of a damaged site of bone, comprising the steps of:
culturing chondrocytes which have been seeded onto a porous body, or differentiating stem cells having chondrogenic differentiation potential which have been seeded onto a porous body into chondrocytes and culturing the chondrocytes; and
implanting the porous body having the cultured chondrocytes.

2. The method for treatment of a damaged site of bone according to claim 1, wherein the stem cells which differentiate into chondrocytes are mesenchymal stem cells.

3. The method for treatment of a damaged site of bone according to claim 2, wherein the mesenchymal stem cells have been harvested from at least one of human bone marrow, human synovium, human fat, and human periosteum.

4. The method for treatment of a damaged site of bone according to claim 1, wherein the chondrocytes which have been seeded onto a porous body are based on chondrocytes which have been harvested from another person or an animal;
the stem cells having chondrogenic differentiation potential which have been seeded onto a porous body are based on stem cells having chondrogenic differentiation potential which have been harvested from another person or an animal; and
the chondrocytes harvested and the stem cells harvested have been subjected to an antigen removal treatment such as decellularization through rapid freezing by liquid nitrogen.

5. The method for treatment of a damaged site of bone according to claim 1, wherein the porous body is formed of a polymer of lactic acid, glycolic acid or caprolactone, or a copolymer thereof.

6. The method for treatment of a damaged site of bone according to claim 1, comprising the step of pulverizing the porous body having the cultured chondrocytes into a powder form,
wherein the implantation step is a step of implanting the porous body that has been made into a powder form in the pulverizing step.
